(19) United States
(12) Patent Application Publication
(10) Pub. No.: US 2023/0175073 A1
(43) Pub. Date: Jun. 8, 2023

(54) ENHANCED CHARACTERIZATION OF BREAST CANCER

(71) Applicant: EPIC SCIENCES, INC., San Diego, CA (US)

(72) Inventors: Ernest LAM, San Diego, CA (US); Martin BLANKFARD, San Diego, CA (US); Nilesh DHARAJIYA, San Diego, CA (US); Richard WENSTRUP, San Diego, CA (US); Ryon GRAF, Encinitas, CA (US)

(73) Assignee: EPIC SCIENCES, INC., San Diego, CA (US)

(21) Appl. No.: 18/076,945

(22) Filed: Dec. 7, 2022

Related U.S. Application Data
(60) Provisional application No. 63/287,014, filed on Dec. 7, 2021.

(51) Int. Cl. C12Q 1/6886; G16B 20/20; G16B 20/10; C12Q 1/6869

(52) U.S. Cl. CPC C12Q 1/6886; G16B 20/20; G16B 20/10; C12Q 1/6869; C12Q 2600/156; C12Q 2600/112

(57) ABSTRACT

Provided herein a method for enhanced assessment of breast cancer in a subject including identification of circulating breast cancer cells in a biological sample and single-cell, whole-genome sequencing of the identified circulating breast cancer cells. Optionally, cell-free tumor DNA is further assessed from the same biological sample or a parallel sample. Also provided are methods of treating a subject with breast cancer using the enhanced assessment to select one or more anti-cancer agents for administering to the subject.

### CROSS-REFERENCE TO RELATED APPLICATIONS

This application claims priority to U.S. Provisional Application No. 63/287,014, filed on Dec. 7, 2021, which is incorporated by reference herein in its entirety.

### BACKGROUND

Breast cancer is the leading cause of cancer mortality (death) among women in most countries in the world, and it is estimated that more than 680,000 breast cancer deaths occurred worldwide in 2020. Furthermore, metastatic breast cancer can arise months or years after a person has completed treatment for early or locally advanced breast cancer. For some patients, metastatic breast cancer is the first cancer diagnosis and is referred to in such circumstances as de novo metastatic breast cancer. In 2020 in the United States alone, it is estimated that more than 168,000 women were living with metastatic breast cancer. The success of treatment and the survival rates for those with recurrent or metastatic breast cancer vary widely, due at least in part to the heterogeneity of breast cancers and challenges in characterizing the type of breast cancer. Historically, breast cancer was diagnosed by acquiring and viewing a surgical biopsy, but such biopsies are invasive, can miss or return an insufficient amount of the tumor or can return only one tissue type from a tumor that is heterogeneous. Thus, surgical biopsies can fail to provide the needed accuracy and sensitivity. More recently, liquid biopsies that assess circulating tumor cells (CTCs) or cell-free, circulating-tumor DNA (cfDNA or ctDNA) have emerged as a less invasive means of diagnosing and assessing breast cancers. Inter- and intra-patient heterogeneity has made it challenging to define a standard liquid biopsy platform. To date, most liquid biopsy tests look only at ctDNA.

### SUMMARY

The present invention includes an enhanced assessment of breast cancer in a subject, optionally, from a single biological sample from the subject, and providing an assessment of circulating tumor cells along with genomic sequencing of the identified circulating tumor cells without a need for cell enrichment, depletion, or microfluidic manipulation. The subject may have or be suspected of having recurrent or metastatic breast cancer. Optionally, cell free DNA is further assessed from the non-cellular portion of the biological sample. The assay thus provides at least two and optionally three assessments, which a clinician can evaluate in order to determine (1) whether the patient's cancer is a recurrence or metastasis of breast cancer and (2) what specific characteristics the breast cancer has. Both determinations can be useful in developing a treatment plan tailored to the specific needs of the subject.

Thus, provided herein is a method of characterizing breast cancer in a subject. The method comprises detecting circulating tumor cells positive for a breast cancer marker in a non-enriched biological sample that comprises cells (e.g., non-tumor cells (e.g., white blood cells and various other cells) and circulating tumor cells). To detect circulating tumor cells, if present in the sample, nucleated cells from the biological sample are first placed on a solid support (i.e., a surface that can be viewed with a scanning microscope, such as a microscope slide). Then, five markers in the nucleated cells are immunofluorescently labelled, wherein the first marker is a nuclear marker, wherein the second marker is an endothelial cell marker, wherein the third marker is a white blood cell marker, wherein the fourth marker is an epithelial marker, and wherein the fifth marker is a cancer marker (e.g., a breast cancer marker). Optionally, the cancer marker is associated with a targeted therapy. Using fluorescent scanning microscopy, labeled nucleated cells on the solid support are identified as circulating tumor cells if they have intact nuclei, are positive for the epithelial marker and are negative for both the endothelial marker and the white blood cell marker. The cells identified as circulating tumor cells are further determined to be breast cancer cells if they are positive for the breast cancer marker or markers. One or more cells identified as circulating tumor cells (e.g., a cell positive for a breast cancer marker) are isolated and used for single-cell, whole-genome sequencing to further characterize the cancer in the subject. For example, single-cell, whole-genome sequencing can be performed. Single-cell, whole-genome sequencing optionally includes determining in each of the at least one circulating tumor cell the presence of large-scale state transitions (LSTs) and copy number variation (CNV) in one or more breast cancer genes in the subject and/or determining discordance of gene sequences in a plurality of circulating tumor cells to identify heterogenous clones in the subject.

Optionally, the cell-free fraction of the non-enriched biological sample from the subject is further analyzed to characterize cell-free DNA by isolating and sequencing cell-free DNA from the biological sample and sequencing the cell-free DNA to characterize the cell free DNA genomic content.

The methods described herein can be used to determine subsequent treatment steps for a subject with recurrent or metastatic breast cancer. Thus, provided herein is a method of treating a subject with recurrent or metastatic breast cancer using the methods described above to identify and characterize the breast cancer and administering to the subject one or more anti-cancer agents specific for the type of breast cancer characterized in the subject.

The details of one or more embodiments are set forth in the description below. Other features, objects, and advantages will be apparent from the description and from the claims.

### BRIEF DESCRIPTION OF THE DRAWINGS

FIG. 1 shows a flow chart of analysis of a single biological sample with analysis of circulating tumor cells shown on the left and analysis of cell-free, circulating-tumor DNA show on the right.

FIG. 2 is a schematic showing the genomic workflow for performing single-cell sequencing on one or more of the cells identified as circulating tumor cells (CTCs).

FIG. 3 is a schematic showing the genomic workflow for processing cell-free, circulating-tumor DNA.

### DETAILED DESCRIPTION

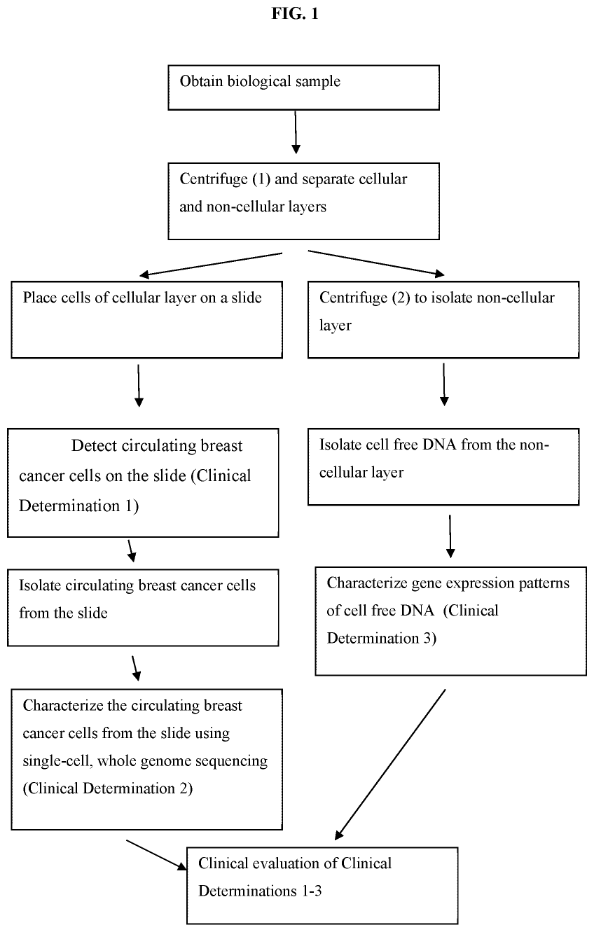

The present method provides an enhanced characterization of breast cancer cells. The enhanced characterization provides reliable analysis by combining (1) identification and assessment of circulating tumor cells (CTCs) and a determination of whether they are positive for certain breast cancer phenotypic markers; (2) single-cell, full genomic sequencing of DNA (ctcDNA) from the circulating tumor cells; and, optionally, (3) an assessment of cell-free, circulating-tumor DNA (ctDNA or cfDNA). The enhanced characterization can optionally be performed on a single biological sample derived from a subject, e.g., a single blood draw sometimes described as a liquid biopsy. The enhanced characterization provides treating physicians with identification and characterization of breast cancer in a subject, which can help guide drug treatment of breast cancer in the subject. FIG. 1 shows the flow chart for one embodiment of the method, including the assessment of circulating tumor cells (CTCs) and single-cell, full genomic sequencing of DNA from a plurality of circulating tumor cells (ctcDNA) on the left and the assessment of cell-free, circulating-tumor DNA (ctDNA or cfDNA) from the sane blood sample on the right.

Taken together, the results of the enhanced analysis can confirm the recurrence or metastasis of breast cancer by identifying the cancer of origin as breast cancer and can characterize the phenotype and genomic characteristics of the subject's cancer. Such a determination allows a more informed and personalized approach to treatment.

### Methods of Characterizing Breast Cancer

Provided herein is a method of characterizing breast cancer in a subject with or suspected of having recurrent or metastatic breast cancer. As used herein, recurrent breast cancer refers to a cancer that recurs in or near the original site, whereas metastatic breast cancer refers to a cancer that has spread to other parts of the body. Thus, a subject may have been treated for breast cancer but subsequently presents with a lump or a pain suggesting a tumor or presents with symptoms associated with bone, liver, lung, or brain cancer. Enhanced characterization of the cancer cells in each case allows the treating clinician to better select optimum treatment.

The enhanced characterization provided by the method accounts for the heterogeneity of breast cancer tumors and provides a comprehensive characterization of circulating tumor cells as well as cell-free, circulating-tumor DNA. The term “circulating tumor cell” or CTC refers to any cancer cell found in a subject's sample. The term “ctcDNA” refers to DNA of a circulating tumor cell. The terms “cell-free DNA,” “cfDNA,” refer to DNA isolated from non-cellular fraction of the sample, and the terms “circulating-tumor DNA, and “ctDNA” refers to the fraction of the cfDNA determined to be of cancer origin.

### Assessment of Circulating Tumor Cells (CTCs) Positive for One or More Breast Cancer Markers

The first step of the method comprises detecting circulating tumor cells in the sample and determining which of these CTCs are positive for one or more breast cancer markers in a biological sample from the subject. The biological sample need not be enriched for tumor cells as enrichment techniques invariably introduces biases. The biological sample is any sample that contains circulating tumor cells and non-tumor cells and can be selected from the group consisting of whole blood, stool bone marrow, pleural fluid, peritoneal fluid, cerebrospinal spinal fluid, urine, saliva, and bronchial washes. In one aspect, the sample is a blood sample, including, for example, whole blood or any fraction or component thereof. A blood sample, suitable for use with the present invention may be extracted from any source known that includes blood cells or components thereof, such as venous or arterial blood. The biological sample may be obtained and processed using routine clinical methods (e.g., procedures for drawing and processing whole blood). The biological sample is optionally centrifuged to create a cellular fraction and a non-cellular fraction. The cellular fraction is then processed by placing nucleated cells (e.g., cells from the buffy coat of a centrifuged blood sample or cells that remain after lysis of red blood cells) on a solid support, which can be used for viewing the cells using a scanning microscope, whereas the non-cellular fraction (e.g., plasma) is optionally processed for detection and characterization of cell-free DNA as described below. The solid support upon which the nucleated cells from the biological sample are placed may be coated with a compound that promotes electrostatic interaction of biological material to the support. A variety of substrate materials are well known in the art and suitable for use with the present invention. Such materials can be selected from the group consisting of glass; organoplastic such as polycarbonate and polymethylmethacrylate, polyolefin; polyamide; polyester; silicone; polyurethane; epoxy; acrylics; polyacrylate; polyester; polysulfone; polymethacrylate; polycarbonate; PEEK; polyimide; polystyrene; and fluoropolymer. In an exemplary aspect, the solid support can be a plate, a slide, or a dish. Slides may include one or more active areas defined on the surface thereof. An active field, as used herein, is intended to include areas in which the slide has been chemically or electrically treated, such as with a biologically interactive coating, for example to promote the adhesion of cells to the slide. For example, the slide may be treated such that the surface is positively charged which allows for cells to be anchored to the surface though the electrostatic adhesion of a negatively charged cell. The slide may include from 1 to any number of active areas depending on the size of the slide and the intended application.

After the nucleated cells are placed on the solid support, the cells are labelled (e.g., immunofluorescently labelled) for about five markers. The first marker is a nuclear marker. The nuclear marker is selected from any nuclear stain or marker. Examples of nuclear stains include 4′,6-diamidino-2-phenylindole (DAPI), Hoechst, propidium iodide. The second marker is an endothelial cell marker. Examples of endothelial markers include CD31, CD34, CD54, CD61, CD62E (E-Selectin), CD105 (Endoglin), CD106 (VCAM-1), CD144 (VE-Cadherin), CD146 (MUC18, Mel-CAM), CD201 (EPCR), CD202b (Tie2/Tek), and CD309 (VEGFR2-Flk-1), Podoplanin, and VEGFR3. The third marker is a white blood cell marker. By way of example, the white blood cell marker can be CD45, CD16, or CD19. The fourth marker is an epithelial marker, which is optionally a cytokeratin or any set thereof selected from cytokeratin 1, 4, 5, 6, 7, 8, 10, 13, 18, and 19; CD24; CD44R, CD49f; CD66a; CD75; CD104; CD121a; CD133; CD167; and CD326. The fifth marker is a breast cancer marker, such as, but not limited to, human epithelial growth factor receptor 2 (HER2), estrogen receptor (ER), progesterone receptor (PR), other breast cancer antigens, or any combination thereof. By way of example, the breast cancer marker can be HER2, ER, or both HER2 and ER.

It should be noted that fewer than five markers may be detected, or more than five markers may be detected based on the specific goals of the test and/or the specific needs of the subject being tested. For example, additional markers, such as PD-L1 and TROP2, may offer other druggable targets. Thus, an additional marker or a different set of five or fewer markers may be useful. Additionally, more than one type of marker of each type could be assessed. For example, more than one nuclear marker, more than one endothelial marker, more than one white blood cell marker, or than one epithelial marker, and/or more than one breast cancer marker can be used.

Certain nuclear stains, such as DAPI, Hoechst, and propidium iodide, auto-fluoresce and need not be further tagged with a fluorophore. For markers lacking auto-fluorescence, immunofluorescent labeling can be performed using fluorophore-labelled antibodies, e.g., a first labelled antibody for an endothelial cell (e.g., CD31), a second labeled antibody for a white blood cell marker (e.g., CD45), a third labeled antibody for an epithelial cell marker (e.g., a cytokeratin or a set of cytokeratins), and a fourth labeled antibody for HER2, ER, PR, or other breast cancer antigens (e.g., mucin 1, carcinoembryonic antigen, carbohydrate antigens (Tn, TF, STn), p53, TERT, and WT1. Alternatively, one or more of the markers could be labeled using an unlabeled primary antibody specific for the marker and labeled secondary antibody that binds to the unlabeled primary antibody. As another alternative, one or more of the markers could be labeled using an unlabeled primary antibody specific for the marker, an unlabeled secondary antibody that selectively binds to the unlabeled primary antibody, and a labelled tertiary antibody that binds to the unlabeled secondary antibody. Additionally biotinylated primary antibodies and streptavidin systems can be utilized for detection. As used herein, the term “antibody” includes intact polyclonal or monoclonal antibodies, single chain antibodies, or antibody fragments, such as Fab, Fab′, and F(ab′)2, scFv, Fv, dsFv diabody, and Fd fragments as well as combinations of such antibodies or fragments. Methods for generating fluorescently labeled antibodies are well known in the art, for example, fluorescent molecules may be bound to an immunoglobulin either directly or indirectly by using an intermediate functional group. Each of the tagged markers are fluorescently labelled with a unique tag such that the five markers can be discerned. Fluorescent tags include, but are not limited to, ethidium bromide, fluorescein, fluorescein isothiocyanate, Alexa Fluor, R-phycoerythrin, Texas Red, green fluorescent protein, rhodamine, mCherry, yellow fluorescent protein, blue fluorescent protein, cyan fluorescent protein, allophycocyanin, mScarlet, fluorescein isothiocyanate (FITC), Oregon Green., tetrarhodamine isothiocynate (TRITC), Cy3, Cy5, Alexa Fluor 647, Alexa Fluor 555, Alexa Fluor 488). Fluorescent tags are selected to allow visualization and discrimination of multiple signals on the same slide and/or within the same cell using different colors and/or wavelengths.

Using fluorescent scanning microscopy, the labeled nucleated cells on the solid support (e.g., a slide) are identified as circulating tumor cells if they have intact nuclei, are positive for the epithelial marker and are negative for the endothelial marker and the white blood cell marker. The cells identified as circulating tumor cells are further determined to be breast cancer cells if they are positive for the breast cancer marker or markers. Detecting circulating tumor cells and circulating tumor cells positive for a breast cancer marker comprises analysis of the morphology and fluorescent labeling of cell images of the nucleated cells. As used herein, “image” referred to an image, such as a digital image, of a sample including various cells, such as CTCs. Typically, a sample image is an image of all or a portion of a sample slide having cells adhered to its surface and optionally stained with one or more detectable markers. Circulating tumor cells can be identified from the images using Epic Sciences BRIA algorithm. See, e.g., Werner et al. (2015), Analytical Validation and Capabilities of the Epic CTC Platform: Enrichment-Free Circulating Tumour Cell Detection and Characterization, J. Circ. Biomarkers 4:3 and U.S. Pat. Nos.: 10,613,089; 10,545,151; and 10,254,286. Detection and localization of the breast cancer marker in each of the identified circulating tumor cells is then performed to further identify the cells as breast cancer cells.

### Single-Cell, Whole-Genome Sequencing to Characterize Breast Cancer Cells

One or more cells identified as circulating tumor cell (e.g., one or more cells positive for the breast cancer marker or negative for one or more breast cancer markers) are isolated and used for single-cell, whole-genome sequencing to characterize the breast cancer in the subject. Isolation optionally involves subjecting the coordinates of the classified cell or cells to a realignment calibration using Calypso software to enable manual isolation of the selected circulating tumor cell. The isolated circulating tumor cell marker is then placed in a single cell isolation system (SCIS). Each SCIS is sequenced, optionally, following amplification (e.g., by whole genome amplification). At least one isolated circulating tumor cell is subjected to single-cell, whole genomic sequencing. However, given the heterogeneity of tumors, more than one circulating tumor cell can be subjected to single-cell, whole genomic sequencing. For example, at four to ten cells may be isolated and sequenced. Thus, 1, 2, 3, 4, 5, 6, 7, 8, 9, 10, or more cells may be isolated and sequenced using single-cell, whole genomic sequencing.

Single-cell, whole-genome sequencing can be performed to confirm the presence of a neoplasm, to characterize the tumor cells, to identify the presence of biomarkers, and/or to identify heterogenous clones. The single-cell, whole-genome sequencing step optionally includes determining in each of the isolated circulating tumor cells the presence of large-scale state transitions (LSTs) and copy number variation (CNV) in one or more breast cancer genes in the subject. Optionally, the CNV determination involves calculating a z-score for calling deletion or amplification of one or more breast cancer genes, such as, but not limited to, the ERBB2/HER2 gene. This step is particularly important for detection of triple negative breast cancer where the CTC is negative for breast cancer markers HER2, ER, and PR. In such case, by way of example, triple negative breast cancer is identified when copy number losses are consistent across the entire genome (with the most frequent variants affecting chromosomes 4 and 5; lack of total gain of the 1q arm).

The single-cell, whole-genome sequencing can optionally include determining discordance of gene sequences in a plurality of at least two circulating tumor cells to identify heterogenous clones in the subject. As used herein discordance refers to a mismatch in biomarker status using, for example, single-cell sequencing in a sample from a single subject. Discordance can be based for example, on the presence of absence of a biomarker (such as Her2+versus Her2−, ER+ versus ER−, PR+ versus PR−, PIK3CA+versus PIK3CA−, or combinations thereof). Differences across biomarkers can include copy number variations (CNVs) or single nucleotide variations (SNVs). Discordance across tumor cells in a sample for the same subject indicates heterogeneity (i.e., at least two different clones are present in the sample).

### Characterization of Cell-Free, Circulating-Tumor DNA

As an optional third prong in the assay method described herein, the method can comprise characterizing cell-free DNA from the non-enriched biological sample, along with identifying CTCs and single cell, whole genomic sequencing of at least one of the identified CTCs. As shown in FIG. 1, a single biological sample can be separated into a non-cellular fraction in order to characterize the cell-free DNA. The “non-cellular fraction” refers to a portion of the sample that is devoid or nearly devoid of cells. A non-cellular fraction can be obtained by methods known in the art, including for example by centrifugation to separate the biological sample into a cellular and non-cellular portion. To ensure the non-cellular portion is devoid or nearly devoid of cells, the method can comprise multiple centrifugation steps and/or filtration steps. By way of example, if the biological sample is a blood sample, the sample can be centrifuged to create a plasma layer, which is carefully separated from the buffy coat and red blood cell layers. The plasma is optionally centrifuged again and/or filtered to remove any residual cells.

The method characterizing the cell-free DNA includes the steps of isolating the cell free DNA from the biological sample and sequencing the cell-free DNA to characterize the cell-free DNA genomic content and pattern of gene expression. Characterizing the cell-free DNA optionally involves assessing the presence of genomic variants (small variants, small insertions and deletions, fusions, and copy number variants) in breast cancer relevant regions. Thus, characterization can include identification of individual gene variants relevant to cancer (e.g., ERBB2/HER2 gene) or multiple gene signatures of tumor mutational burden, and/or microsatellite instability. These parameters are determined using next generation sequencing and targeted sequencing technology.

### Methods of Treating Breast Cancer

Also provided herein is a method of treating a subject with recurrent or metastatic breast cancer. The treatment of breast cancer is complicated by the heterogeneity of cancer types. For example, HER2 positive, ER positive, and/or PR positive tumors are responsive to certain therapeutic agents that triple negative breast cancers are not. Thus robust, reliable characterization of breast cancer in a subject provides the opportunity for better selection of treatment and better predictability of a successful outcome. For this reason, the method of treatment first requires characterizing the breast cancer in a biological sample from the subject as described above by first detecting circulating tumor cells positive for a breast cancer marker in the biological sample by placing nucleated cells from the biological sample on a solid support, immunofluorescently labeling five markers in the nucleated cells, wherein the first marker is a nuclear marker, wherein the second marker is an endothelial cell marker, wherein the third marker is a white blood cell marker, wherein the fourth marker is an epithelial marker, and wherein the fifth marker is a breast cancer marker, and identifying labeled nucleated cells on the solid support using fluorescent scanning microscopy to identify circulating tumor cells (i.e., cells with intact nuclei and are positive for the epithelial marker and negative for the endothelial marker and the white blood cell marker; and determining whether the circulating tumor cells are positive for the breast cancer marker to detect circulating tumor cells positive for the breast cancer marker. Next, the method requires characterizing the breast cancer in the subject with single-cell, whole genome sequencing by isolating at least one of the identified circulating tumor cells and performing amplification and sequencing of the isolated circulating tumor cell(s). Once the breast cancer is characterized, one of skill in the art could select an optimal treatment agent for the subject and administer to the subject one or more anti-cancer agents specific for the characterized breast cancer in the subject. Further as described above, the biological sample optionally can be separated into a cell-free fraction for isolation and sequencing of the cell-free DNA in order to character the cell-free DNA based on the genomic content (e.g., the presence of gene variants in a breast cancer relevant region, tumor mutational burden, microsatellite instability, or any combination thereof). Alternatively, a first biological sample from the subject can be processed for analysis of circulating tumor cells and a second sample processed for cell-free tumor DNA.

Based on the laboratory findings, one of skill in the art upon determining the breast cancer is HER2 positive could administer a HER2 specific treatment such as monoclonal antibodies or conjugates thereof (e.g., Trastuzumab, Pertuzumab, Margetuximab, Ado-trastuzumab emtansine, and/or Fam-trastuzumab deruxtecan), kinase inhibitors (e.g., Lapatinib, Neratinib, and/or Tucatinib) or any combination thereof. Similarly, if one of skill in the art determines the breast cancer is hormone receptor positive (ER and/or PR positive), a specific treatment for hormone receptor positive cancer can be selected including, for example, CDK4/6 inhibitors (e.g., Palbociclib, Ribociclib, and/or Abemaciclib), mTOR inhibitors (e.g., Everolimus), and/or PI3K inhibitors (e.g., Alpelisib). Additional agents can also be provided. For example, if additional genetic mutations are identified in either circulating tumor cells or cell-free tumor DNA, e.g., BRCA1 or 2 mutations, agents such as PARP inhibitors (e.g., Olaparib and/or Talazoparib). Importantly, when heterogeneous cell types are found, patients can be treated for both cell populations, and treatments can be prioritized, combined, and/or sequenced based on clinical factors, probable distal metastasis source, and likelihood of response.

One of skill in the art could combine one or more of these agents with each other or with other chemotherapeutic agents.

As used herein, patient or subject may be used interchangeably and can refer to a subject with or at risk of developing breast cancer. The term patient or subject includes human and veterinary subjects. The subject can be a vertebrate, more specifically a mammal (e.g., a human, horse, cat, dog, cow, pig, sheep, goat, mouse, rabbit, rat, and guinea pig). The term does not denote a particular age or sex.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

The examples below are intended to further illustrate certain aspects of the methods and compositions described herein and are not intended to limit the scope of the claims.

### EXAMPLES

### Example 1: Detection of Circulating Tumor Cells Positive for a Breast Cancer Marker

Nucleated cells from blood samples were deposited onto glass slides and, optionally, stored at −80° C. until analysis. Each slide was then subjected to nuclear staining with DAPI, as well as staining with fluorescently labelled antibodies specific to CD45, multiple cytokeratins (CK), and CD31. CTCs were identified as CK+, CD45−, CD31− and DAPI+ with intact nucleus and morphology consistent with cancer. In the present assay, fluorescently labelled antibodies specific for breast cancer protein biomarkers HER2, ER, or PR were used to stain the slides in order to further characterize whether the CTCs were breast cancer cells. Stained slides were then imaged, and CTCs were detected and analyzed using proprietary digital pathology algorithms. See, e.g., Werner et al. (2015), Analytical Validation and Capabilities of the Epic CTC Platform: Enrichment-Free Circulating Tumour Cell Detection and Characterization, J. Circ. Biomarkers 4:3 and U.S. Pat. Nos. 10,613,089; 10,545,151; and 10,254,286.

### Example 2: Single Cell, Whole-Genome Assessment of Circulating Tumor Cells

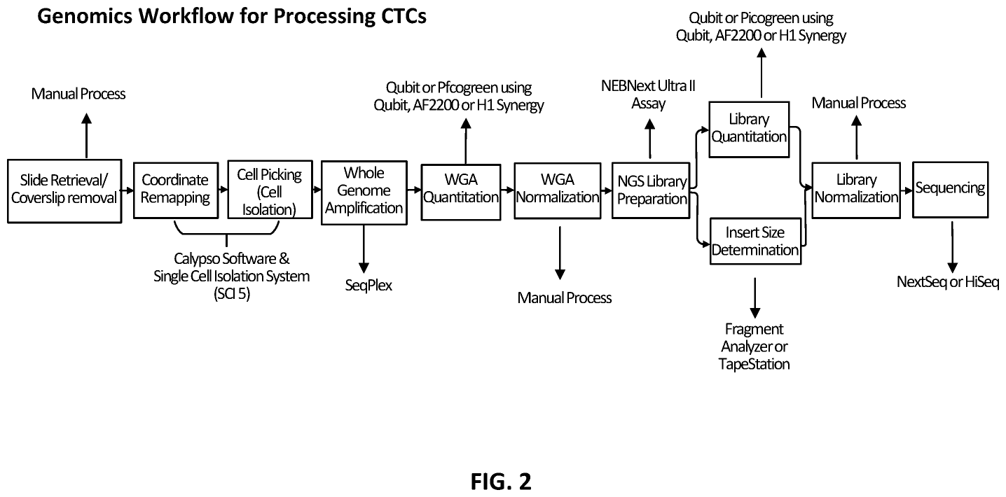

For single cell analysis, all nucleated cells from a whole blood sample were stained for immunofluorescence microscopy, then individually analyzed for marker expression and morphology using proprietary Epic Sciences BRIA algorithm to identify circulating tumor cells (CTCs) as described in Example 1. Additionally, detection and localization of HER2 and/or ER protein expression were further used to identify breast cancer CTCs. The coordinates of the classified cells were subjected to a realignment calibration using Calypso software to enable manual CTC isolation on the single cell isolation system (SCIS). Cells then underwent a manual preparation for whole genome amplification (WGA) using the Sigma SeqPlex Assay kit. The amplified DNA was quantified using a fluorometric intercalating dye (e.g., Picogreen or Qubit) prior to normalization. 100-200 ng of the normalized product was then processed using New England Biolabs NEBNext Ultra II NGS assay. The final libraries were quantified with Picogreen or Qubit. The insert size of the libraries were assessed using either a Fragment Analyzer or TapeStation automated CE platforms. Libraries were normalized to 2 nM, before preparation for sequencing on the Illumina NextSeq or HiSeq platforms. For CTC genomic analysis, the number of large-scale state transitions (LSTs) was determined by analyzing the read coverage profiles and tallying large, significant copy number changes indicative of cancer. Clinically significant copy number variants in genes of interest were detected using a z-score based metric, which indicated how much the read coverage in the cell of interest deviates from the typical read coverage in a set of normal genomes. Single cell sequencing was performed for up to 10 circulating breast cancer cells from each patient. See FIG. 2.

### Example 3: Assessment of Cell-Free Tumor DNA

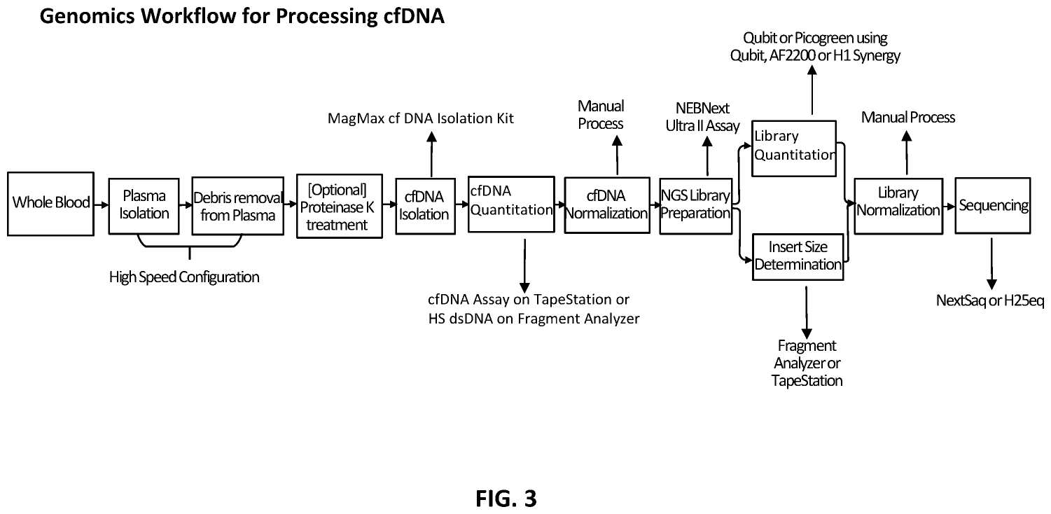

Whole blood collected in Streck Cell-Free BCT tubes was centrifuged at 2000×g for 10 minutes to separate plasma. The isolated plasma underwent a second high-speed centrifugation to remove residual blood and cellular debris. The obtained plasma was treated with Proteinase K. Circulating cell-free DNA (ctDNA) was isolated from the plasma using MagMax cfDNA isolation kit. The DNA was then quantified using either the Cell Free DNA screentape on the TapeStation or High Sensitivity dsDNA assay on the Fragment Analyzer. Normalized samples were then processed using New England Biolabs NEBNext Ultra II NGS assay. The final libraries were quantified with Picogreen or Qubit. The insert size of the libraries was assessed using either a Fragment Analyzer or TapeStation automated CE platforms. Libraries were normalized before preparation for sequencing on the Illumina NovaSeq platform. Extracted ctDNA from blood plasma was then interrogated by a targeted Next Generation Sequencing (NGS) panel of 56 clinically relevant genes for detection of low frequency single nucleotide variants (SNVs), small insertions/deletions (indels), fusions, and copy number variation (CNV). In addition to variant calls, the NGS method reports a tumor mutational burden (TMB) score and microsatellite instability (MSI) status for ctDNA. See FIG. 3.

### Example 4: Clinical Relevance

A physician reviews the results of Example 1 and 2 and optionally of Example 3 in order to make a determination of whether the subject has cancer, whether the cancer is breast cancer, and whether the breast cancer is HER2 and/or ER positive. In three cases where surgical biopsies were available from the same subjects, the liquid biopsy results showed circulating tumor cells positive for HER2 and CTC and ctDNA sequencing positive for cancer, but surgical biopsies were negative for HER2. Physician analysis evaluated the liquid biopsy results and determined the subject had HER2 positive breast cancer, which would have been missed with surgical biopsy alone.

The dispersions, products, and methods of the appended claims are not limited in scope by the specific dispersions, products, and methods described herein, which are intended as illustrations of a few aspects of the claims and any dispersions, products, and methods that are functionally equivalent are intended to fall within the scope of the claims. Various modifications of the dispersions, products, and methods in addition to those shown and described herein are intended to fall within the scope of the appended claims. Further, while only certain representative dispersion materials and method steps disclosed herein are specifically described, other combinations of the dispersion materials and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed.

1. A method of characterizing breast cancer in a subject, comprising
(a) detecting circulating tumor cells positive for a breast cancer marker in a biological sample from the subject, wherein the biological sample is not enriched, comprising
(1) placing nucleated cells from the biological sample on a solid support,
(2) immunofluorescently labeling five markers in the nucleated cells, wherein the first marker is a nuclear marker, wherein the second marker is an endothelial cell marker, wherein the third marker is a white blood cell marker, wherein the fourth marker is an epithelial marker, and wherein the fifth marker is a breast cancer marker,
(3) identifying labeled nucleated cells on the solid support using fluorescent scanning microscopy to identify circulating tumor cells, wherein the circulating tumor cells have intact nuclei, are positive for the epithelial marker and are negative for the endothelial marker and the white blood cell marker, and
(4) determining whether the cells identified in step (3) as circulating tumor cells are positive for the breast cancer marker;
(b) isolating at least one identified circulating tumor cell from step (a); and
(c) performing single-cell, whole-genome sequencing of the at least one isolated circulating tumor cell from step (b) to obtain genomic information regarding the breast cancer in the subject.

2. The method of claim 1, wherein the biological sample is a blood sample.

3. The method of claim 1, wherein the nuclear marker is DAPI or Hoechst.

4. The method of claim 1, wherein the epithelial marker is one or more cytokeratins.

5. The method of claim 1, wherein the white blood cell marker is CD45.

6. The method of claim 1, wherein the endothelial marker is CD31.

7. The method of claim 1, wherein the breast cancer marker is a human epithelial growth factor receptor 2 (HER2), estrogen receptor (ER), or both HER2 and ER.

8. The method of claim 1, wherein the detecting step comprises analysis of the morphology and fluorescent labeling of cell images of the nucleated cells.

9. The method of claim 1, wherein the single-cell, whole-genome sequencing comprises determining in each of the at least one circulating tumor cell the presence of large-scale state transitions (LSTs) and copy number variation (CNV) in one or more breast cancer genes in the subject.

10. The method of claim 9, wherein the CNV determination comprises calculating a z-score for amplification of ERBB2/HER2 genes.

11. The method of claim 1, wherein the single-cell, whole-genome sequencing comprises determining discordance of one or more biomarkers in a plurality of circulating tumor cells to identify heterogenous clones in the subject.

12. The method of claim 1, further comprising characterizing cell-free DNA from the non-enriched biological sample, comprising
(d) isolating cell-free DNA from the biological sample, and
(e) sequencing genes of interest in the cell-free DNA to characterize the genomic variants in the circulating-tumor DNA.

13. The method of claim 12, wherein the cell free DNA is characterized by assessing presence of gene variants relevant to breast cancer, tumor mutational burden, microsatellite instability, or any combination thereof.

14. The method of claim 1, wherein characterizing breast cancer in a subject identifies the cancer of origin as breast cancer.

15. The method of claim 1, wherein the subject has or is suspected of having recurrent breast cancer.

16. The method of claim 1, wherein the subject has or is suspected of having metastatic breast cancer.

17. A method of treating a subject with breast cancer, comprising
(a) detecting circulating tumor cells positive for a breast cancer marker in a biological sample from the subject, wherein the biological sample is not enriched, comprising
(1) placing nucleated cells from the biological sample on a solid support,
(2) immunofluorescently labeling five markers in the nucleated cells, wherein the first marker is a nuclear marker, wherein the second marker is an endothelial cell marker, wherein the third marker is a white blood cell marker, wherein the fourth marker is an epithelial marker, and wherein the fifth marker is a breast cancer marker,
(3) identifying labeled nucleated cells on the solid support using fluorescent scanning microscopy to identify circulating tumor cells, wherein the circulating tumor cells have intact nuclei, are positive for the epithelial marker and are negative for the endothelial marker and the white blood cell marker, and
(4) determining whether the cells identified in step (3) as circulating tumor cells are positive for the breast cancer marker;
(b) isolating at least one identified circulating tumor cell from step (a);
(c) performing single-cell, whole-genome sequencing of the at least one isolated circulating tumor cell from step (b) to obtain genomic information regarding the breast cancer in the subject; and
(d) administering to the subject one or more anti-cancer agents specific for the characterized breast cancer in the subject.

18. The method of claim 17, wherein the subject has a recurrent breast cancer.

19. The method of claim 17, wherein the subject has metastatic breast cancer.

20. The method of claim 17, wherein the biological sample is a blood sample.

21. The method of claim 17, wherein the nuclear marker is DAPI of Hoechst.

22. The method of claim 17, wherein the epithelial marker is one or more cytokeratins.

23. The method of claim 17, wherein the white blood cell marker is CD45.

24. The method of claim 17, wherein the endothelial marker is CD31.

25. The method of claim 17, wherein the breast cancer marker is a human epithelial growth factor receptor 2 (HER2), estrogen receptor (ER), or both HER2 and ER.

26. The method of claim 17, wherein the detecting step comprises analysis of the morphology and fluorescent labeling of cell images of the nucleated cells.

27. The method of claim 17, wherein the single-cell, whole-genome sequencing comprises determining in each of the at least one circulating tumor cell the presence of large-scale state transitions (LSTs) and copy number variation (CNV) in one or more breast cancer genes in the subject.

28. The method of claim 27, wherein the CNV determination comprises calculating a z-score for amplification of ERBB2/HER2 genes.

29. The method of claim 17, further comprising detecting one or more breast cancer markers in cell-free DNA from the non-enriched biological sample, comprising
(e) isolating cell-free DNA from the biological sample, and
(f) sequencing genes of interest in the cell-free DNA to characterize the genomic variants in the circulating-tumor DNA.

30. The method of claim 29, wherein the cell free DNA is characterized by assessing presence of gene variants relevant to breast cancer, tumor mutational burden, microsatellite instability, or any combination thereof.

31. The method of claim 17, wherein characterizing breast cancer in a subject identifies the cancer of origin as breast cancer.

32. The method of claim 17, wherein the subject has or is suspected of having recurrent breast cancer.

33. The method of claim 17, wherein the subject has or is suspected of having metastatic breast cancer.
